(19) United States
(12) Patent Application Publication
(10) Pub. No.: US 2009/0048515 A1
(43) Pub. Date: Feb. 19, 2009

(54) BIOPSY PLANNING SYSTEM

(76) Inventors: Jasjit S. Suri, Roseville, CA (US); Dinesh Kumar, Grass Valley, CA (US); Yujun Guo, Grass Valley, CA (US); Ramkrishnan Narayanan, Nevada City, CA (US)

Correspondence Address: MARSH, FISCHMANN & BREYFOGLE LLP, 8055 East Tufts Avenue, Suite 450, Denver, CO, 80237, US

(21) Appl. No.: 11/838,518

(22) Filed: Aug. 14, 2007

(51) Int. Cl. A61B 10/02; A61B 8/13

(52) U.S. Cl. 600/443; 128/898; 600/562

(57) ABSTRACT

Guided biopsy is a commonly used method to remove suspicious tissues from an internal organ for pathological tests so that malignancy can be established. Provided herein are systems and methods (i.e., utilities) that allow for automated application of one or more predefined biopsy target plans to an acquired medical image including without limitation, an ultrasound prostate image. Due to different shapes and sizes of prostates as well as orientation of prostate with respect to an ultrasound probe during image, acquisition a simple prostate model (e.g., ellipse) with a fixed plan may not be sufficient. Accordingly, it has been determined that a deformable shape model with integrated biopsy target locations/sites may be fit to a prostate image to provide improved automated biopsy targeting.

### FIELD OF INVENTION

The present invention is directed at image guided biopsy procedures. More specifically, the present invention is directed at applying predetermined biopsy locations/plans to medical images to reduce the time required to perform a biopsy procedure and/or improve the accuracy of such a procedure.

### BACKGROUND OF THE INVENTION

Prostate cancer is the second leading cause of death among males in the USA. However, it, is often curable if detected at an early stage. Accordingly, early detection and treatment is important. In general, a biopsy is recommended when a patient shows high levels of PSA (Prostate Specific Antigen), which is an indicator of prostate cancer or the patient has an abnormal physical exam (e.g., digital rectal exam). Ultrasound has been the main imaging modality for prostate related applications due its inexpensiveness, ease of use, and ability to scan in real-time during biopsy and treatment.

Transrectal Ultrasound (TRUS) Guided Prostate Biopsy is a standard procedure for obtaining biopsy samples with ultrasound guidance. In such a procedure an end-fire or side-fire ultrasound probe is used that generates a 2D image plane. For biopsy sampling, most probes require a needle set placed through a guide tube parallel to the axis of the probe and the needle set can be extended beyond the end of the probe to obtain a biopsy sample. During the procedure, the urologist inserts the ultrasound probe into the rectum, and moves the probe until specific area of the prostate to be sampled is identified in the live ultrasound scan. The urologist then moves or bends the probe upward, pointing the biopsy needle channel or biopsy needle set guide at the targeted area of the prostate. A needle set is inserted through the needle channel or guide, pushed through the rectum wall and into the prostate. The needle is then fired using La biopsy gun and tissue samples are collected. Usually, multiple samples are taken from different areas of the prostate, typically between 6 and 18 samples.

There have been efforts to plan optimal locations for prostate biopsies. However problems arise due to the 2-D nature of ultrasound image. That is, the plan is defined in a 3-D frame, of reference but the manual guidance of needle by the urologist is done based on guidance using a 2-D image. With the advent of techniques for reconstructing a 3-D volume from a sequence of 2-D ultrasound images, a user is now able to plan a biopsy procedure in a 3-D volume corresponding to the prostate shape of the individual patient. In such a scenario, the urologist, acquires a series of 2-D ultrasound images of the prostate, which are reconstructed into a 3-D image-volume by the system. The user then plans the target sites for collection of tissue using the 3-D image and, then proceeds to collect the samples from the planned sites. However, selection of the target sites is still done manually and depends upon the skill of the user in selecting target sites while also adding to the time of the procedure. This adds extra burden on the workflow and affects the sensitivity and specificity of the, procedure.

An alternative to the manual approach is to arrange 6, 12, 16 or 18 core biopsy sites 100 on a biopsy site model 100 (see FIG. 1A) and overlay the biopsy site model onto an object (e.g., prostate 104) of a real data set, (e.g., a 3-D image). Such a procedure provides a rudimentary automated biopsy planning system. However, such an automated fitting procedure does not take shape and topology of the real data set (e.g., prostate image) into consideration while loading the target sites in the planning system.

In medical imaging systems, one major challenge lies in handling the changing shapes of the anatomies due to growth, hydration, diseases or in response to the treatment during therapeutic procedures. Thus the main limitation of the current automated planning systems lies in their inability to handle the changes in shape of the imaged, object. In many instances, simply applying a biopsy site model 102 to the object 104 results in one or more biopsy locations 106 being located outside of the boundary of the, object 104. See FIG. 1B. That is, by not accounting for changes in the shape of an object from individual to individual, overlaid biopsy models may fail to correctly locate biopsy positions. Further, many medical imaging systems result in images without sufficient information in one or more occluded planes. This further complicates the image planning and guidance systems, which require *putting the 3D core locations where biopsies need to be drawn from.

A number of systems or devices have been proposed for the purpose of better targeting of biopsies. For instance, one system includes an ultrasonic transrectal probe and an ultrasonic transurethral probe where each probe is in operative communication with an integrated patient support platform and an integrated expert system. The integrated expert system collects data transmitted by sensors in the transrectal and transurethral probes and produces level-of-suspicion mapping of the prostate gland with cancer probability assessments for areas contained within the level-of-suspicion mapping. Generally, previous systems, have required specialized equipment, and do not make use of existing ultrasound systems and technology or have required fusion of multiple modalities (e.g., MRI, CT, ultrasound) and/or the implant of fiducial markers or seeds as landmarks or references. Finally, previous systems have failed to utilize prior information specific to current patient and/or specific to a previously identified group (e.g., demographic group) of patients.

### SUMMARY OF THE INVENTION

Guided biopsy is a commonly used method to remove suspicious tissues from an internal organ for pathological tests so that malignancy can be established. Provided herein are systems and methods (i.e., utilities) that allow for automated application of one or more predefined biopsy target plans to an acquired medical image, including, without limitation, an ultrasound prostate image. In this regard, the utilities may be implemented in software and computer processing systems that are integrated into medical imaging devices and/or that are interconnected to the medical imaging devices and operative to receive data therefrom.

The utilities allow for automatically loading a standard or a customized biopsy plan onto a medical image. Due to different shapes and sizes of prostates as well as orientation of prostate with respect to the ultrasound probe during image acquisition, a simple prostate model (e.g., ellipse) with a fixed plan may not be sufficient. Accordingly, it has been determined that a deformable shape model with integrated biopsy target locations/sites may be fit to a prostate image. Such a shape model may incorporate standard plans (e.g., sextant, plans, etc.) or customized plans based on, for example, demographic information and/or prostate regions known to be susceptible to cancer (e.g., atlas information and/or previous biopsy information). One main advantage of this utility is full automation (e.g., real time application) of standard or other prepared biopsy plans to a prostate image, which improves workflow and reduces time during the biopsy procedure, while being,accurate. The utility is also flexible to allow the users to also add and refer to the saved customized plans.

According to a first aspect, a method for use in applying biopsy target sites to medical prostate images is provided. The method includes obtaining a deformable shape model that is generated from a plurality of prostate images. Use of such a shape model allows for better fitting the model to an acquired prostate image. That is, the shape model, or mean shape of a sample population, allows for better fitting a biopsy plan to different sizes and shapes of prostates as well as orienting the biopsy sites with respect to a currently acquired prostate image. Once the deformable shape model is obtained, a biopsy plan may be identified for use with the shape model. This biopsy plan may then be loaded into the deformable shape model such that the biopsy target locations associated with the plan are registered to locations within the deformable shape model.

In a first arrangement, the biopsy plan may be dynamically loaded into a deformable shape model. For instance, during a prostate imaging procedure, the user may select a biopsy plan that may be loaded into the deformable shape model. Accordingly, the shape model and biopsy plan may then be fit to the prostate image. In another arrangement, the biopsy plan may be loaded into the shape model prior to the procedure. In such an arrangement, a plurality of different biopsy plans may be loaded into shape models such that each shape model includes a specific biopsy plan. Accordingly, such a plurality of deformable shape, models and biopsy plans may be stored in a database for selection by a user.

According to another aspect, a method is provided wherein a plurality of biopsy targeting plans are provided to a user during the prostate imaging procedure. The user may then select one or more of the biopsy targeting plans. Accordingly,, biopsy target locations associated with a selected one or more of the biopsy targeting plans may be applied to the image of the prostate. Furthermore, the method includes outputting a processed image of the prostate, with the biopsy target locations illustrated on the processed image. As will be appreciated, this processed image may allow for guidance of a biopsy device to the 1locations of interest (e.g., biopsy target sites).

In a further arrangement, the plurality of biopsy targeting plans may include previous biopsy information. For instance, such information may include a previous biopsy procedure performed on the patient. This previous biopsy procedure and the locations of previous biopsies may be stored for subsequent use. Accordingly, at a subsequent procedure, the previous biopsy locations associated with the previous procedure may be selected and applied to the processed image. A physician/user may then identify previous locations where biopsies were performed and select new locations and/or apply a new biopsy plan to the processed image.

Such biopsy plans may include, without limitation, conventional plans that provide a predetermined number of locations (e.g., sextant, 8, 12, etc.) as well as customized plans that are based on, for example, statistical information. In this latter regard, it will be appreciated that biopsy plans may be developed based on statistical information associated with known regions in the prostate having increased likelihood of cancer. Applying the biopsy target locations to the image of the prostate may include use of a shape model that allows for adjusting the desired biopsy locations to the individualized characteristics of the prostate of a current patient. In this regard, it may be desirable to utilize a shape model that is generated based on actual prostate images.

### BRIEF DESCRIPTION OF THE DRAWINGS

FIG. 1A illustrates a, prostate biopsy location plan.

FIG. 1B illustrates application of the plan of 1A to a prostate

FIG. 2 illustrates an overall system for acquiring ultrasound images and applying predetermined biopsy plans to that image.

FIG. 3, illustrates an imaging device for use in obtaining an ultrasound image and applying a predetermined biopsy plan to the image.

FIG. 4A illustrates a plurality of two-dimensional images.

FIG. 4B illustrates a three-dimensional image generated from the two-dimensional images of FIG. 4A.

FIG. 5 illustrates a process flow diagram for the deformation of a stored biopsy plan onto a current image.

FIG. 6 illustrates a process for generating a mean shape/model.

FIGS. 7A-C illustrate a predetermined biopsy plan as applied to a shape model, a prostate volume and the shape model applied to the prostate volume, respectively.

FIG. 8 illustrates various zones on a prostate.

FIG. 9 illustrates the implementation of a biopsy plan into a shape model.

FIG. 10 illustrates a process for deforming a shape model to match a current image.

FIG. 11 illustrates a process for mapping reference plan onto current image using thin-plate splines.

FIG. 12 illustrates a screenshot that may be utilized with the presented system.

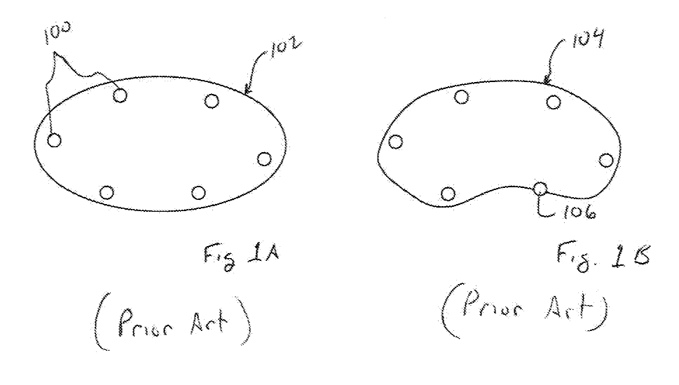

### DETAILED DESCRIPTION

Reference will now be, made to the accompanying drawings, which assist in illustrating the various pertinent features of the present disclosure. Although the present disclosure is described primarily in conjunction with transrectal ultrasound imaging for prostate imaging it should be expressly understood that aspects of the present invention may be applicable to other,medical imaging applications. In this regard, the following description is presented for purposes of illustration and description.

Presented herein are systems and processes (utilities) to aid urologists (or other medical personnel) in planning target sites for biopsy. Generally, the utilities use biopsy site model that may be fit (e.g., warped) to an image of a prostate. Such fitting accounts for differently shaped prostates. These, biopsy shape models may incorporate statistical information regarding various zones within a prostate where the cancer resides and/or probability maps of cancer locations obtained from an expert (histologist) based ground truth selection.

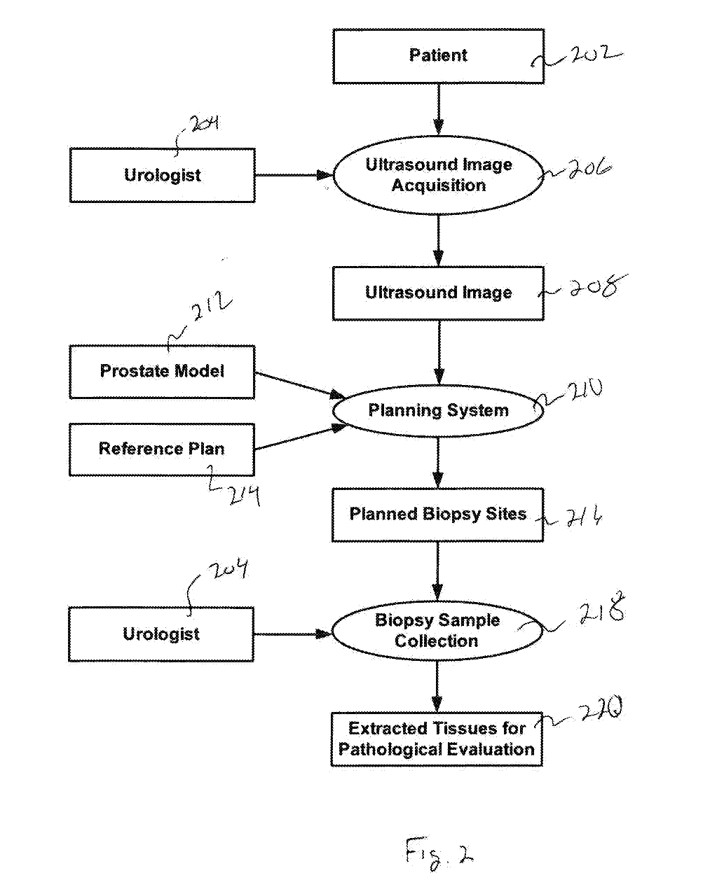

The current invention is aimed at automatic targeted biopsy procedure. FIG. 2 describes an overall process 200 for an ultrasound guided biopsy procedure where an automated biopsy planning system is utilized to locate biopsy locations on/in an ultrasound image. Initially, the patient 202 is positioned by a physician 204 (e.g., on an examination table), and a 3-D image of the prostate is acquired 206 using, for example, a transrectal ultrasound (TRUS) transducer. The resulting 3-D image 208 may either be directly obtained by the TRUS probe or reconstructed on the fly from a sequence of 2-D images obtained through either rotation or translation of TRUS probe, or a, combination of both methods.

After acquiring the image 208, the 3-D volume is observed and target locations are located by a biopsy planning system 210. The planning system 210 utilizes a deformable prostate model 212 and one or more reference plans 214 to automatically locate biopsy locations on the ultrasound image. That is, a number of predetermined standard and/or customized sampling plans 214 are defined and stored in frame of reference of a prostate model 212. During the procedure, the model 212 is deformed into the 3-D volume/image 208 acquired from the patient 202 and the plan 214 is automatically deformed into the new frame of reference. This results in generating an image having planned biopsy sites 216 located thereon and/or therein. The physician 204 may then perform biopsy sample collection 218 at the planned sites to obtain tissue for pathological evaluation. 220. Various portions of the process 200 are discussed herein.

Ultrasound Image Acquisition

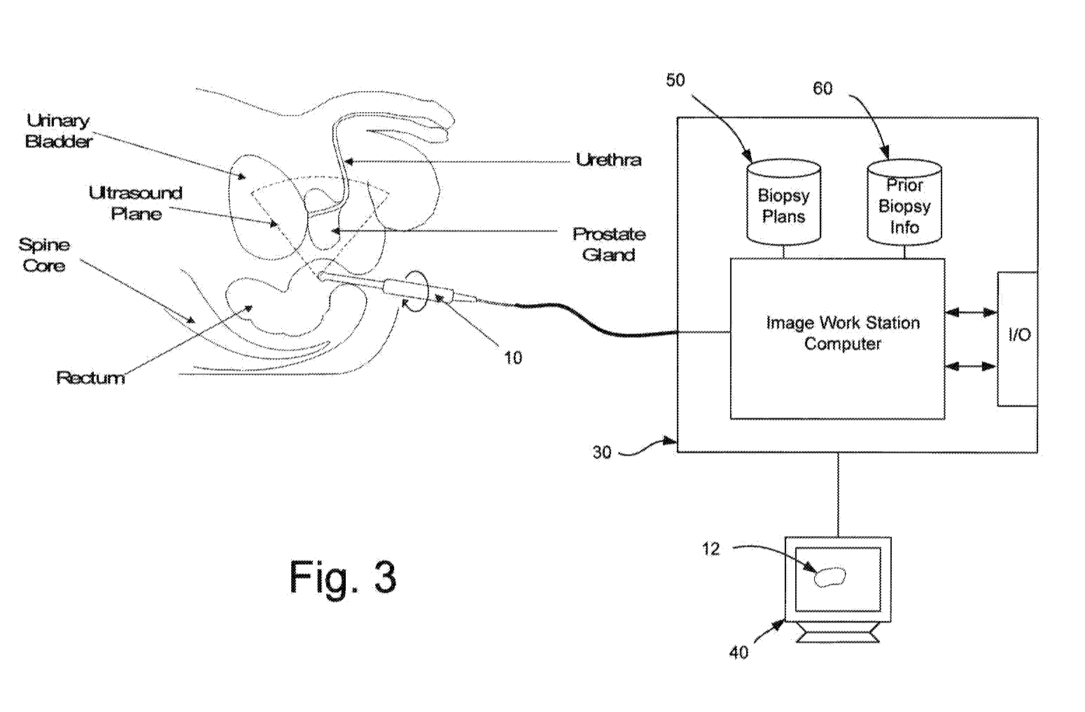

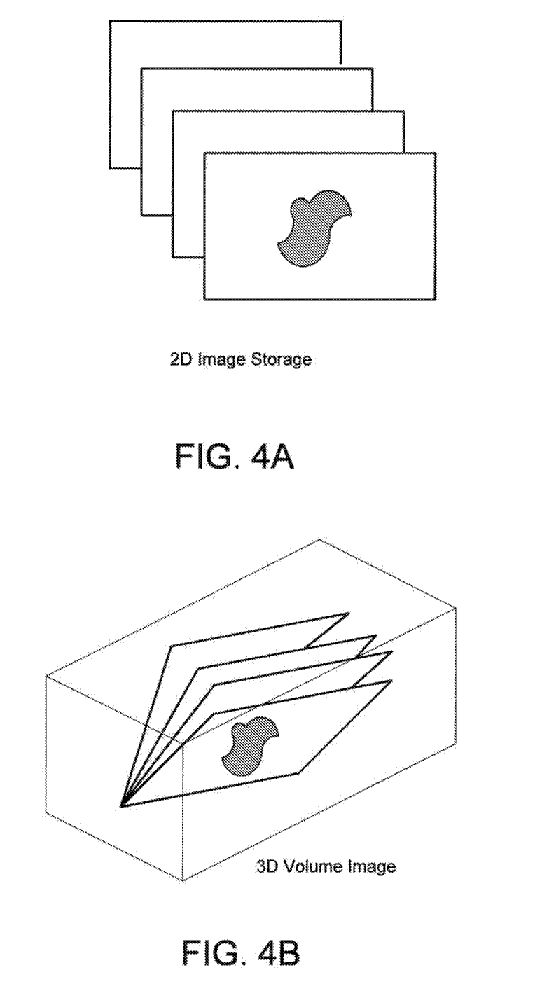

Initially, a 3-D ultrasound image of a prostate of a patient is acquired using, for example a transrectal ultrasound (TRUS) system. See FIG. 3. The acquired images may then be converted to 3-D orthogonal voxel data (e.g., ultrasound volumes) having equal resolution in all three dimensions. The images may be acquired in an appropriate manner. FIG. 3 illustrates a transrectal ultrasound probe 10 being utilized to obtain a plurality of two-dimensional ultrasound images of a prostate 12. As shown, the probe 10 may be operative to automatically scan an area of interest. In such an arrangement, a user may rotate the acquisition end 14 of the ultrasound probe 10 over an area of interest. Accordingly, the probe 10 may acquire plurality of individual images while being, rotated over the, area of interest. See, FIG. 4A. Each of these individual images represents a two-dimensional image. Initially, the stack of such images may be in a polar or curvilinear or any other non-Cartesian coordinate system. In such an instance, it may be beneficial for processing to translate these images into a rectangular coordinate system. In any case, the two-dimensional images may be combined to generate a 3-D image see FIG. 4B. That is, the processing platform 30 of the ultrasound imaging device may receive the 2-D images and generate a 3-D image, which may be output to the physician urologist on a monitor 40. The processing platform 30 also includes a database 50 of biopsy plans. A selected one (or more) of the biopsy plans and/or prior biopsy information 60 (e.g., patient specific information) may be fit to an acquired image to provide biopsy target site locations on the 3-D image output to the monitor 40. One advantage of this process is full automation of stored biopsy plans, which improves work flow and reduces time during the biopsy procedure, while being accurate. The method is flexible to allow the users to also, add and refer to the saved customized plans.

Planning System

In a conventional planning system, the ultrasound image is utilized by a physician to identify target biopsy locations. These target locations are identified based solely on the judgment of the physician. However, this can require a significant amount of time thereby increasing the overall time required for a biopsy procedure. An alternative to the conventional planning approach is using predefined biopsy plans (e.g., 6, 12, 16 or 18 cores sites) with a deformable shape model and fitting the shape model to a prostate image. As discussed herein, the use of a deformable shape model takes into account differences in shape, scale and topology while integrating the target sites into the image.

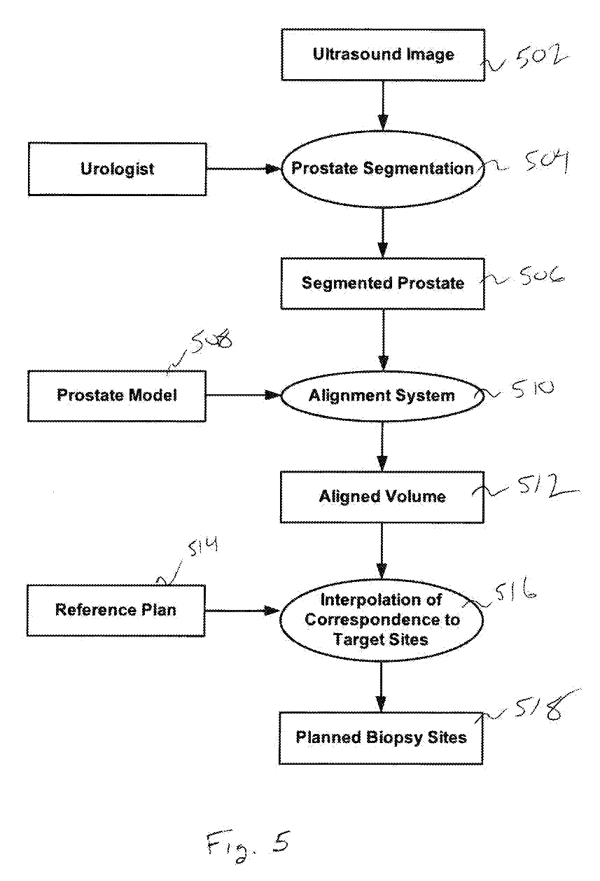

The automatic loading of a predetermined biopsy plan to a current frame of reference can be done in a number of ways. In a general scenario, as illustrated in FIG. 5, an ultrasound image 502 of a patient is acquired and provided to a segmentation processor 504. The segmentation processor generates segmented image. 506. The segmented prostate image 506 and a prostate model 508 are provided to an alignment system 510 which aligns the model and image to a common reference frame to produce an aligned volume 5.12. A reference plan 514 is loaded with the prostate model in an interpolation process 516. That is, a model of the shape of the organ is constructed and the target sites are defined (i.e., loaded) on this model shape. At the time of automatically loading the plan, the model shape can be deformed into the 3-D volume of the target volume to provide planned biopsy sites on the prostate image/volume 518. At such time, a physician may take biopsy samples from the planned sites. See FIG. 2.

For the shape of a prostate, a number of plans may be defined on a simple-shape such as an ellipsoid. The ellipsoid can then be deformed into the shape of the actual organ imaged at the time of image acquisition. The deformation can then be interpolated to deform the target locations into the frame of reference of the 3-D target volume. The deformation may be performed via intensity registration, segmentation of organ followed by surface registration, anatomical, landmark registration or a combination of these methods. Rather than using a simple shape as a prostate model, a mean shape model generated from actual prostate images can provide a number of advantages.

That is, a mean shape of a population defines a shape that has least differences from the population in statistical sense. In addition to using the mean shape, the population shape statistics can be used to deform the shape in ways more meaningful than registration based on just the differences between two images. The next section describes the construction of a mean shape of population and the methods used to compute the statistics over a set of shapes. Once a mean shape has been computed or a model chosen, it is equally important to place the standard or customized plans on this shape.

Shape Model

The first step is the construction of a prostate shape model. While simplistic solutions exist such as assuming a synthetics shape of an ellipsoid or any other surface of revolution, specifically for a prostate shape, computing a mean shape over a number of actual prostate images provides a more meaningful solution. That is use of actual prostate images results in a mean shape that better describes a population (e.g., specific demographic group) compared to picking a synthetic-shape. Plans defined on a mean shape computed from a set of training images are thus more anatomically relevant. Further, the deformation of a synthetic shape does not mimic the actual, anatomical deformation compared to a mean being deformed using population shape statistics where the main modes of variation correspond to the typical deformations characterizing the shape descriptions within the subspace of a shape model generated from actual images. Further, the mean shape is invariant to rotation, scaling and translation and requires the shortest description to fit to the current shape which is assumed to lie within the span of the set of training shapes (e.g., actual prostate images). Thus using a mean shape will generally provides a better solution than an arbitrary image or synthetic model. Such a mean shape, may be generated in a manner similar to the method described in U.S. patent application Ser. No. 11/740,807, entitled, “Improved System and Method for 3-D Biopsy,” the entire contents of which is incorporated by reference.

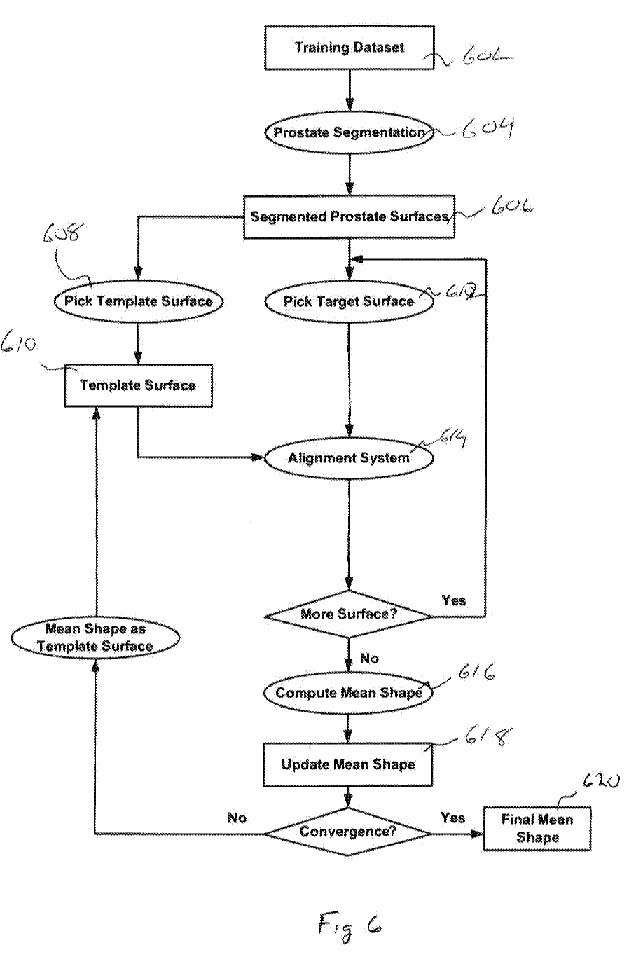

FIG. 6 illustrates a process. 600 for generating a mean shape. The first step is to obtain a number of samples from a population. This is done by scanning the organ (prostate) over a number of subjects and collecting the 3-D (e.g., grayscale) prostate images. That is, a training set 602 is acquired. Next, the prostates in the training set are segmented 604 from the 3-D images/volumes using either expert manual segmentation, a semi-automatic segmentation process such as disclosed in U.S. patent application Ser. No. 11/615,596, entitled, “Object recognition System for Medical Imaging,” the entire contents of which are incorporated by reference or in a fully automatic segmentation approach as described in U.S. patent application Ser. No. 11/833,404, entitled, “Improved Object Recognition System for Medical imaging,” the entire contents of which are incorporated by reference. This generates a set of segmented prostate surfaces 606.

One segmented prostate surface is selected 608 as the tentative template surface 610 or tentative mean shape. Each remaining segmented prostate surface (i.e., target surface) may selected 612 and aligned (e.g., Procrustes aligned) with the template surface 614 to result in a set of aligned shapes with rotation, scaling and translation differences removed. This set of aligned shapes is averaged 616 resulting in a new mean shape 618. The process is repeated until successive iterations of the computed mean shape are nearly identical (i.e, until convergence). This results in a final mean shape 620 for the training data set.

In addition, the shape statistics of the training set may be encapsulated into modes of variation computed via active shape model analysis. In this regard, such shape statistics may be used to drive the registration or even to compute the object boundaries.

Construction of Plans

The next step after construction of a standard shape model (or mean shape) is to define conventional plans (of standard plans) on the shape model. Literature exists on the conventional plans (e.g., sextant biopsy) followed by urologists as, well as on computation of optimal positions for detection of cancer via use of a probabilistic atlas similar to that discussed disclosed in U.S. patent application Ser. No. 11/740,807, entitled, “Improved System and Method for 3-D Biopsy,” as incorporated above. The presented utility is easily extensible to include any new or customized plans.

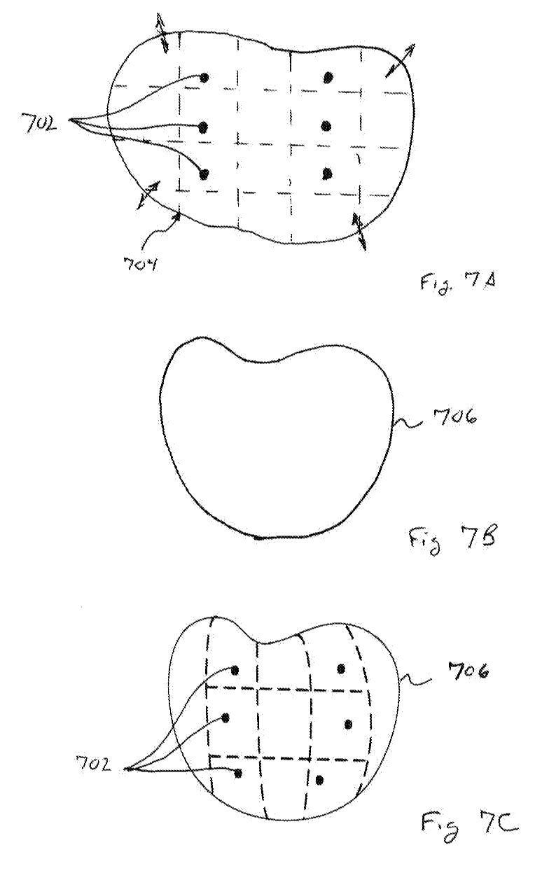

For construction of a conventional plan, there are a number of options manual placement of the plan over the model by an expert, semi-automatic placement of plan through landmark identification by the expert and placement of plan relative to these landmarks, fully automatic placement of plan through automatic landmark detection and automatic placement of points and automatic even distribution of sites in the target plan based on the mean shape. The proposal is general enough to include any of these ideas in construction of the database of standard plans. In addition, optimal plans computed from a probabilistic atlas, may also be used. FIG. 7A illustrates the placement of a sextant biopsy plan including, six biopsy locations 702 into a deformable model 704. Methods,for placing biopsy locations within a model are discussed herein.

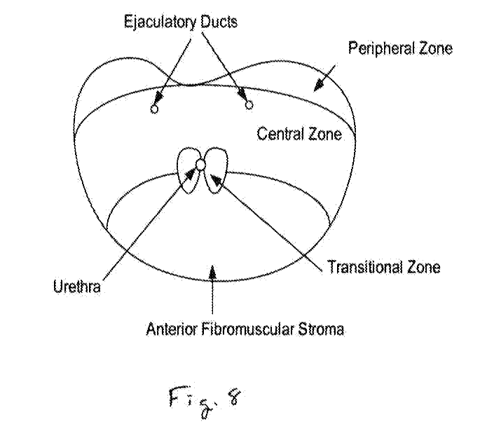

As will be appreciated, different zones of prostate correspond to different prostate anatomy. As shown in FIG. 8, various zones are distributed around the whole prostate, except the central part where the urethra intersects the prostate. Accordingly, it may be desirable to place biopsy sites in different zones of the prostate. For the sextant biopsy plan, there are 3 zones on each side of the prostate. On each side, one zone is set close to the base, one is close to the apex, and the third one is on the middle gland. If more biopsy sites are planned, each of these zones can be further divided into smaller zones, so that more samples will be taken for the biopsy. See, for example, FIG. 7A.

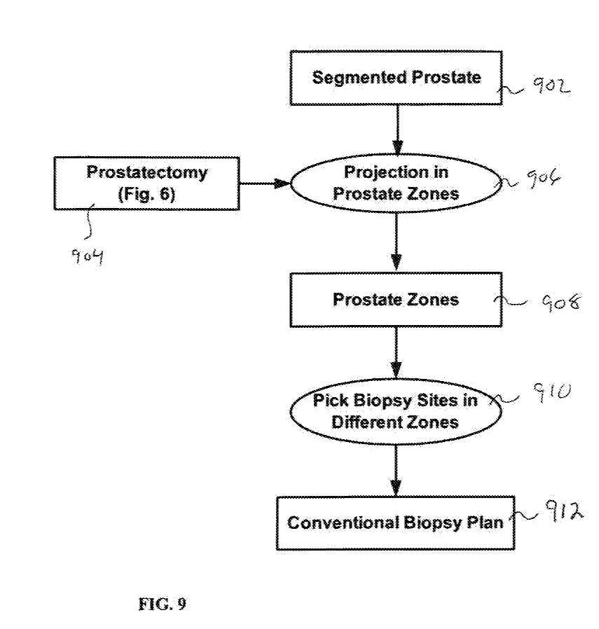

FIG. 9 presents a conventional plan construction system. As illustrated, the segmented prostate images 902 of the training set (See, e.g., FIG. 6) are combined with zonal data 904 taken from removed prostate glands (e.g., prostatectomies). That is, the zonal data is projected 906 into the segmented images such that prostate zones 908i are, defined in the mean shape/model. Biopsy sites are then selected in different zones to define a conventional biopsy plan 912. This plan (e.g., sextant biopsy plan) may then be stored to a database such that a physician may at the time of the biopsy procedure, select the plan for implementation with a current prostate image.

In addition to construction of standard plans (e.g., 8, 12, 16, 20 biopsy locations), additional biopsy plans from previous visit(s) may also be stored by the system. Such previous biopsy plans may be archived together with previous ultrasound scans and corresponding segmented prostate surfaces. Previous biopsy plans can be important, as a urologists may want to revisit previous biopsy sites, or avoid doing biopsy at the same sites. Previous biopsy plans are also an option for use with reference plans.

Loading a Plan in Current Prostate Volume

Loading a plan from the frame of reference of the model into the frame of reference of the target image (the 3-D image volume acquired during the current procedure) requires finding correspondences between the two frames of reference. This can be done using a variety of registration techniques depending upon the available information. Different techniques are discussed below.

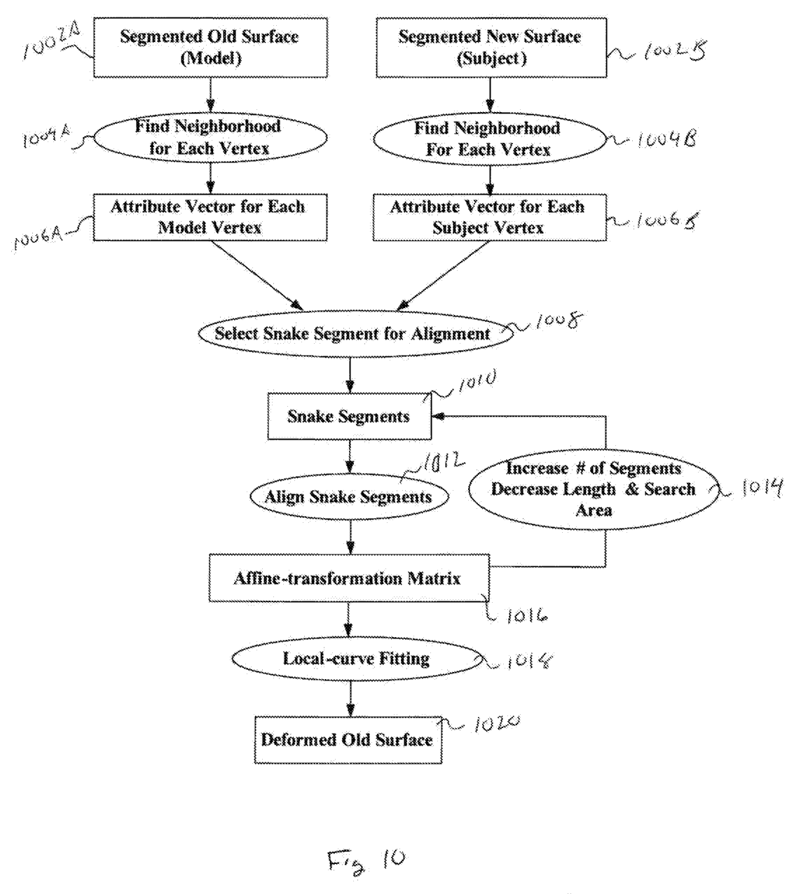

As shown in FIGS. 7A-7C, if the object boundaries from the current prostate volume 706 are available, then the surface of the model 704 (or mean shape) can be registered with the surface of the subject prostate 706. This allows for registering the biopsy locations 702 of the model 7041 with the current prostate volume/image 706. This may be done using a surface registration, technique such as an adaptive focus deformable model. Such an algorithm is illustrated in FIG. 10. Initially, the shape model and subject prostate are segmented 1002A, 1002B. Then for each vertex in the model its neighborhood information is searched 1004A and saved as attribute vector for it 1006A. Also, for each vertex in the subject, its neighborhood information is searched 1004B and saved as attribute vector for it 1006B. A multi-resolution alignment strategy 112 is carried out by sub-sampling a set of snaxels 1008 along the snake contour using initial search length in the neighborhood, and deforming their corresponding snake segments 1010. Such alignment 1012 may be performed using the deforming forces defined between vertices in the model and its closest vertex in the subject, and vice versa. An affine-transformation, matrix 1016 is obtained after the alignment of snake segment. Then the search length, is decreased, therefore increasing the number of snake segments 1014. This alignment procedure is repeat. That is the alignment procedure may be iteratively repeated until maximal number of iterations is reached. A local curve-fitting procedure 1018 is performed to refine the deformation and final deformed old surface 1020 is obtained at the end of alignment procedure. The boundary correspondences obtained as a result of the surface registration can be used to interpolate and deform the plan locations from the boundaries into the target shape and displayed on the 3-D image volume See, e.g., FIG. 5.

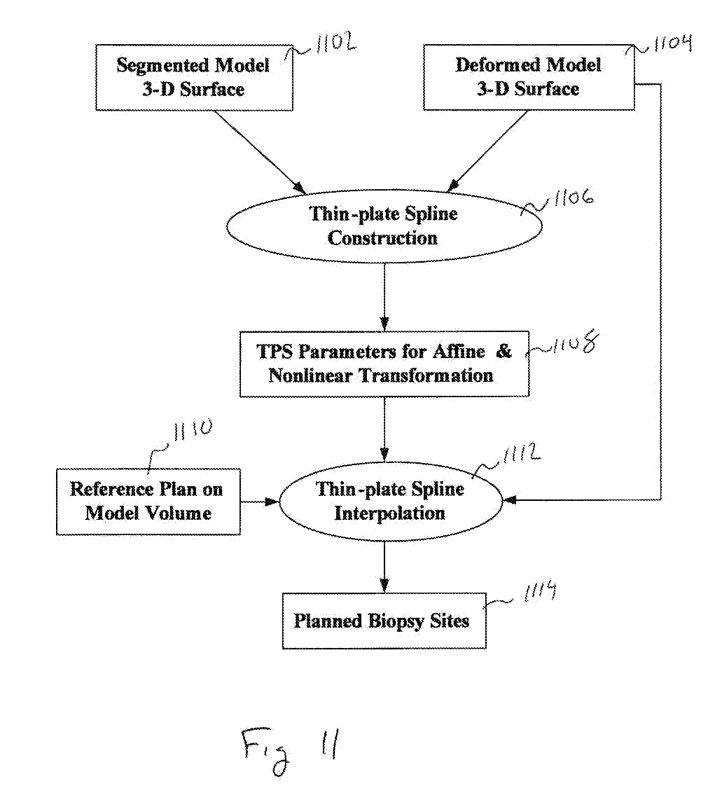

The interpolation may be done using an elastically deformable model such as, for example, using a thin-plate spline based interpolation or any boundary elements based or finite elements based method. FIG. 11 illustrates the interpolation procedure using thin-plate splines. The inputs are the model surface 1102 and the deformed model surface 1104 from the alignment process. Since the model surface 1102 and its deformed version 1104 has one-to-one correspondence for each of its vertex, a global transformation based on thin-plate splines can be constructed 1106. The parameters for both affine and nonlinear parts in the thin-plate splines transformation 1108 are obtained after the construction. Through those parameters, the biopsy sites identified in the reference plan 1110 can be mapped onto current image using thin-plate spline interpolation 1112, therefore planned biopsy sites 1114 can be identified.

Alternatively, the two surfaces may be registered together using the shape statistics obtained after computing the mean shape from a set of samples. The coefficients for the modes of variations are computed hierarchically such that they deform the model shape into the target shape using a boundary matching cost criterion. The deformation at boundaries can be, used to compute deformation at the plan such that the plan is deformed from the coordinate system of the mean shape into the coordinate system of the 3-D volume. If the boundaries of the object from the current 3-D scanned volume are available, the shape model can be used to compute the segmentation. This is done by deforming the mean shape into the frame of reference of the target image. A linear combination of the basis vectors spanning the lower dimensional shape space added to the mean shape provides us with a typical shape. The basis vectors in this shape space account for most of the variance in the entire training set. The coefficients of the basis vectors can be optimized such that the shape obtained is maximally similar to the shape in the target image.

Intensity based registration also may be performed such that the registration directly provides solution over the entire image volume and the deformation computed at the planned locations are deformed into the 3-D grayscale image volume. Further, shape statistics may be directly used to find the deformation by allowing the shape to deform through the modes of variations computed earlier such that the mean shape deforms into the object shape. This is essentially same as performing the segmentation, but the deformation obtained at the boundaries can directly be used to compute the deformation at the planned locations. The interpolation may follow any of the methods discussed above.

Adding a New Plan

Previous sections describe methods used in deforming the shape model and biopsy sites defined in model into the frame of reference of a 3-D prostate volume. The same method can be used to add a customized plan for adding, an optimized plan from a probabilistic atlas. This can be done identifying biopsy locations of a customized plan on the 3-D volume of the shape model. In case of atlas, this represents the frame of reference of the atlas. The volume (or the atlas space) is deformed into the shape of model using a method identical to method described in relation to FIG. 10. This is essentially the same method with the correspondence now being defined in the opposite direction instead. As a result,, the customized plan is, now deformed into the frame of reference of the mean shape. This plan can then be saved in the database of plans available.

The new plan now resides along with other standard plans in the same frame of reference, e.g., the frame of reference of the mean shape. For future reference, the user may now select this plan, from the list and the proposed method then treats it like any of the standard plans already loaded.

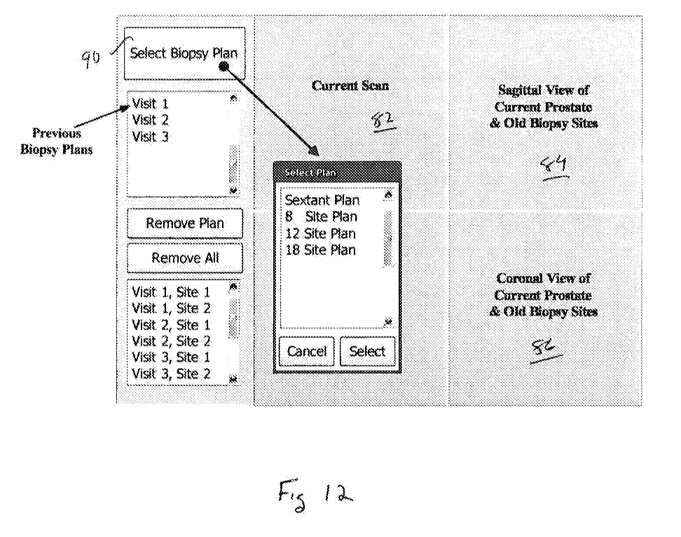

FIG. 12 illustrates a graphical user interface that may be utilized in conjunction with the imaging device of FIG. 3. In this regard, the graphical user interface may be displayed on the monitor 40, illustrated in FIG. 3. As shown, the graphical user interface 80 includes a number of display areas 82,, 84, 86 that allow for displaying the current image and/or displaying the current image in different views and/or displaying prior images and/or prior biopsy information onto the current image. Display area 82 is typically utilized for live ultrasound image acquisition. Moreover, the graphical user interface 80 includes user selectable biopsy plans 90. In this regard, a user may select a biopsy plan from a menu of biopsy plans and have that biopsy plan applied to a current image. As will be appreciated, such selection and application to the image may be done in substantially real time. That is, the previously stored plans that are integrated with a shape model may be fit to the current image and thereby provide biopsy sites at desired locations therein. In addition, a plurality of previous biopsy plans and biopsy results may be accessible for viewing. As shown in FIG. 3, such prior biopsy information may be stored in prior biopsy information database, 60.

The overall planning system, which allows for applying predetermined biopsy plans to current medical image, allows for increasing the accuracy and speed in which a biopsy procedure may be performed. Further, while simplistic solutions exist for applying simplistic (e.g., sextant biopsy plans) to a prostate image, computing a mean shape over a number of actual prostate images provides a more meaningful solution. That is, the mean shape describes a population better than a simplistic/synthetic shape, and any plans defined on the mean shape of actual images provides improved anatomical information in comparison to synthetic shapes. Further, the deformation of a synthetic shape often does not mimic the actual anatomical deformation in comparison to a mean shape being deformed using population shape statistics. That is, the mean shape is the closest to the population in a statistical sense and, therefore, typically requires, on average,, smaller deformation to fit to the current shape. Such smaller deformations are typically associated with smaller registration errors and thereby provide Ea better fit solution.

Another advantage of the present system is that using information from previous visits in a repeat biopsy may help a physician better interpret a current scan. In this regard, the physician may select to revisit or avoid previous biopsy plans presented on a current volume.

Importantly, the system allows a user to select available biopsy plans from a reference plan list. This allows a physician to rapidly implement a plan they feel best suited for a current patient. In any case, a selected reference plan may be projected onto a current volume after accurate alignment with/integration into the prostate model. Further, use of the deformable shape model takes into consideration changes in prostate shape from patient to patient. Finally, it will be appreciated that the system allows a user/physician to add new plans or edit standard plans, allowing for full customization of biopsy procedure.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are. within the scope of the present invention. The embodiments described hereinabove are further intended to explain best modes known of practicing the invention and to enable others skilled in, the art to utilize the invention in such, or other embodiments and with various modifications required by the particular application(s) or use(s) of the present invention. It is intended that the appended claims be construed to include alternative embodiments to the extent permitted by the prior art.

What is claimed:

1. A method for use in applying biopsy target sites to a medical prostate image, comprising:
obtaining a deformable shape model, wherein said shape model is generated from a plurality of prostate images;
identifying a biopsy plan including multiple biopsy target locations; and
loading said biopsy plan into said deformable shape model, wherein said biopsy target locations are registered,to locations within said deformable shape model.

2. The method of claim 1, further comprising:
fitting said shape model and said registered biopsy target locations to an image of a prostate of a patient, wherein said biopsy target locations are displayed on said image.

3. The method of claim 2, wherein said steps of loading and fitting are performed in conjunction with acquiring said image of said prostate.

4. The method of claim 1 further comprising:
loading statistical information into said shape model.

5. The method of claim 1, wherein, said shape model and said registered target biopsy locations define a biopsy model, further comprising:
storing said biopsy model to a biopsy model database.

6. The method of claim 5, further comprising:
repeating said identifying loading and storing steps for a plurality of biopsy plans, wherein said biopsy model database includes a plurality of biopsy models.

7. A method for use in applying biopsy target sites to a medical prostate image, comprising:
obtaining a medical image of a prostate of a patient;
receiving an input selecting at least one of a plurality of biopsy targeting plans, wherein each of said biopsy targeting plans includes one or more biopsy target locations;
applying said biopsy target locations to said image of said prostate; and
outputting a processed image of said prostate with said biopsy target locations illustrated on said processed image.

8. The method of claim 7, further comprising:
storing said processed image of said prostate to a database; wherein said processed image may be accessed at a subsequent time.

9. The method of claim 7, further comprising:
outputting a previous prostate image of said patient with said processed image, wherein said previous image was acquired from said patient during a previous procedure.

10. The method of claim 9, further comprising:
aligning said previous image with said processed image.

11. The method of claim 10, further comprising:
identifying previous biopsy locations on said processed image.

12. The method of claim 7, wherein applying said biopsy target locations to said image of said prostate further comprises:
fitting said shape model associated with a selected one of said biopsy targeting plans to said image of said prostate, wherein said biopsy target locations are aligned with said image.

13. The method of claim 12, further comprising:
loading said selected biopsy plan into said shape model.

14. The method of claim 12, wherein said shape model comprises a deformable shape model generated from a plurality of prostate images.

15. A method for use in applying biopsy target sites to a medical prostate image, comprising:
outputting a first image of a prostate of a patient;
providing a plurality of predefined biopsy plans, said biopsy plans each including one or more biopsy target locations;
loading a selected one of said predefined biopsy plans into a deformable shape model, wherein said biopsy target locations of said selected biopsy plan are, registered to locations within said deformable shape model,
fitting said deformable shape model to said first image; and
outputting a second image of said prostate, wherein said second image includes said biopsy target locations.

16. The method of claim 15, wherein said deform able shape model is generated from a plurality of prostate images.

17. The method of claim 15, wherein providing a plurality of predefined biopsy plans comprises:
providing at least a first convention biopsy plan and providing, previous biopsy location information for said patient.
